(19) United States
(12) Patent Application Publication
(10) Pub. No.: US 2024/0269411 A1
(43) Pub. Date: Aug. 15, 2024

(54) ENDOTRACHEAL TUBE WITH ULTRASOUND-VISIBLE DOUBLE CUFF

(71) Applicant: HAMAD MEDICAL CORPORATION, Doha (QA)

(72) Inventor: Nabil Shallik, Doha (QA)

(21) Appl. No.: 18/566,520

(22) PCT Filed: Jun. 3, 2022
(86) PCT No.: PCT/QA2022/050011

§ 371 Date: Dec. 1, 2023

Related U.S. Application Data
(60) Provisional application No. 63/196,440, filed on Jun. 3, 2021.

(51) Int. Cl. A61M 16/04

(52) U.S. Cl. CPC A61M 16/0411; A61M 16/0438; A61M 16/044; A61M 16/0456; A61M 2205/3331; A61M 2205/583; A61M 2209/045; A61M 2210/065

(57) ABSTRACT

An endotracheal tube system includes an endotracheal tube, a first expandable bag disposed on the endotracheal tube, and a second expandable bag surrounding the first expandable bag. The second expandable bag is configured to contain a contrast agent. The first expandable bag is disposed on a distal portion of the endotracheal tube.

### CROSS REFERENCE TO RELATED APPLICATION

The present application claims priority to U.S. Provisional Patent Application No. 63/196,440, filed Jun. 3, 2021, the disclosure of which is incorporated into this specification by reference in its entirety.

### BACKGROUND

A significant number of patients undergoing general anesthesia need endotracheal intubation and/or laryngeal mask airway insertion. Endotracheal intubation confirmation can be performed with a variety of typical techniques, such as chest auscultation, end-tidal capnography, such as end-tidal carbon dioxide (ETCO₂) waveform monitoring, observing a patient's chest rising, and/or a chest x-ray. Misplacement of endotracheal tubes during intubation, however, is a potential complication that may cause increased operative morbidity and mortality.

### SUMMARY

A new and innovative endotracheal tube system and method of inserting the same into a patient's trachea is provided. In some examples, an endotracheal tube system may include an endotracheal tube, a first expandable bag disposed on the endotracheal tube, and a second expandable bag surrounding the first expandable bag, wherein the second expandable bag is configured to contain a contrast agent.

In an aspect of the present disclosure, which may be combined with any other aspect listed herein unless specified otherwise, the first expandable bag may be disposed on a distal portion of the endotracheal tube.

In an aspect of the present disclosure, which may be combined with any other aspect listed herein unless specified otherwise, the first expandable bag may be configured to contain air.

In an aspect of the present disclosure, which may be combined with any other aspect listed herein unless specified otherwise, the contrast agent may be ultrasound-visible.

In an aspect of the present disclosure, which may be combined with any other aspect listed herein unless specified otherwise, the contrast agent may comprise agitated water or saline.

In an aspect of the present disclosure, which may be combined with any other aspect listed herein unless specified otherwise, the endotracheal tube system may further include an injection device, wherein the injection device may include: a body; and one or more ports on or in the body, wherein the body may include: an inner space; and a flexible surface facing the inner space, wherein the injection device may be in a deflated state when the flexible surface is pressed down towards the inner space.

In an aspect of the present disclosure, which may be combined with any other aspect listed herein unless specified otherwise, the injection device may be in an inflated state when a pressure pressing the flexible surface down towards the inner space is removed.

In an aspect of the present disclosure, which may be combined with any other aspect listed herein unless specified otherwise, the one or more ports may include: a first port configured to be in fluid communication with a source of the contrast agent; a second port configured to be in fluid communication with the second expandable bag; and a third port configured to be in fluid communication with the first expandable bag.

In an aspect of the present disclosure, which may be combined with any other aspect listed herein unless specified otherwise, the injection device may be configured to deliver the contrast agent to the second expandable bag while the injection device is transitioned from the deflated state to an inflated state.

In an aspect of the present disclosure, which may be combined with any other aspect listed herein unless specified otherwise, the second port and the third port may be connected to the second expandable bag and the first expandable bag, respectively, while ultrasonography is performed on a target patient.

In an aspect of the present disclosure, which may be combined with any other aspect listed herein unless specified otherwise, the first and second expandable bags may be transparent.

In some examples, a method of inserting the endotracheal tube system into a patient's trachea is provided. The method may include: inserting the endotracheal tube in the patient's trachea; inflating the first expandable bag by injecting a first material into the first expandable bag; inflating the second expandable bag by injecting the contrast agent into the second expandable bag; and partially deflating the first expandable bag.

In an aspect of the present disclosure, which may be combined with any other aspect listed herein unless specified otherwise, the method may further include checking a position of the endotracheal tube using an extracorporeal scanner, wherein the contrast agent may be visible when viewed using the extracorporeal scanner.

In an aspect of the present disclosure, which may be combined with any other aspect listed herein unless specified otherwise, the extracorporeal scanner may include an ultrasound probe.

In an aspect of the present disclosure, which may be combined with any other aspect listed herein unless specified otherwise, the method may further include, responsive to checking the position, deflating the second expandable bag by removing the contrast agent from the second expandable bag, and inflating the first expandable bag, thereby stabilizing the first expandable bag.

In an aspect of the present disclosure, which may be combined with any other aspect listed herein unless specified otherwise, the first material may include air.

In an aspect of the present disclosure, which may be combined with any other aspect listed herein unless specified otherwise, the first expandable bag may be partially deflated while the second expandable bag is inflated, thereby preventing overpressure on the patient's trachea.

In an aspect of the present disclosure, which may be combined with any other aspect listed herein unless specified otherwise, the first expandable bag may be disposed on a distal portion of the endotracheal tube.

Additional features and advantages of the disclosed method and apparatus are described in, and will be apparent from, the following Detailed Description and the Figures. The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE DRAWINGS

FIG. 1 is a diagram of an endotracheal tube inserted into a patient's trachea.

FIG. 2 is a diagram of an endotracheal tube system according to an example embodiment of the present disclosure.

FIG. 3 is a diagram of an example injection device of the endotracheal tube system of FIG. 2, the injection device being in a deflated state.

FIG. 4 is a diagram of an example injection device of the endotracheal tube system of FIG. 2, the injection device being in an inflated state.

FIGS. 5A-F are diagrams of various configurations of example injection devices according to an example embodiment of the present disclosure.

FIG. 6 is a cross-sectional view of an injection device according to an example embodiment of the present disclosure.

FIG. 7 is a cross-sectional view of an injection device according to another example embodiment of the present disclosure.

FIGS. 8A-L are diagrams illustrating a method of inserting the endotracheal tube system of FIG. 2 into a patient's trachea and confirming the location of the endotracheal tube system.

### DETAILED DESCRIPTION

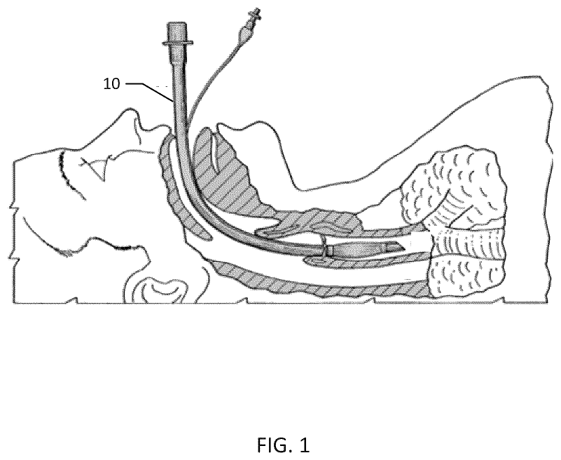

FIG. 1 illustrates a diagram of an endotracheal tube 10 inserted into a patient's trachea. As discussed above, a number of patients undergoing general anesthesia may need endotracheal intubation. However, misplacement of the endotracheal tubes during intubation may cause a serious complication. Since the endotracheal tube 10 is inserted into a patient's body, it is not easy to detect the location of the endotracheal tube during intubation.

Ultrasound to a patient's armamentarium may allow rapid visualization of structures that are typically not apparent until performing direct laryngoscopy and can therefore have diagnostic value for identifying esophageal intubation. For instance, with optimal sensitivity and specificity, ultrasonography can be a valuable supplement to patient airway assessment and endotracheal intubation confirmation, especially in situations where capnography and/or chest auscultation may be unreliable.

Ultrasound-guided intubation can, however, be limited in that there is no direct visualization of the endotracheal tube cuff using typical ultrasound-guided intubation techniques. As such, the lack of direct visualization can make a medical professional unable to confirm the location of the tip of the endotracheal tube, which can cause misplacement of the endotracheal tube during intubation.

Aspects of the present disclosure may address the above-discussed deficiencies in the related system. The present application provides a new and innovative endotracheal tube system and method of inserting the endotracheal tube system into a patient's trachea and confirming the location of the endotracheal tube system.

In some examples, an endotracheal tube system may include double balloons which may enable a medical professional to visualize a typical tracheal tube balloon, for example, via ultrasound. The first balloon may be filled with air. The second balloon surrounding the first balloon may be filled with a contrast agent that can be seen, for example, via an ultrasound probe on the front of a patient's neck to confirm a position of the endotracheal tube. For example, the contrast agent may be agitated water or saline. In some examples, the second balloon may be filled with water or saline and may be externally connected to an air-filled syringe. Air from the syringe may be introduced into the second balloon to create agitated water or saline. The agitated saline (with huge number of very small air bubbles) may be very cheap, safe, and easy to be used as a contrast material to the ultrasound beam.

Aspects of the present disclosure may enable visualization of a tracheal tube balloon without the use of coatings or other materials on the endotracheal tube that could be irritating for a patient's airways. Aspects of the present disclosure may also help a user confirm the final position of the tube in the trachea above the carina and prevent the tube from migrating to one main bronchus.

An example method of using the endotracheal tube may include inserting a tracheal tube in the patient's trachea. The first, or inner, balloon may then be inflated with air. The second, or outer, balloon may then be filled with water or saline. Air may then be introduced into the water or saline-filled outer balloon to create agitated water or saline in the outer balloon. Air may be deflated from the first balloon to regulate the pressure in the first balloon. After confirmation of the intubation and the position of the endotracheal tube in the patient's trachea, the agitated water or saline can be purged from the second balloon. Air may be reinjected into the first balloon to stabilize the first balloon.

As described in the above example method, water may need to be injected into and purged from the outer balloon, while at the same time the inner balloon is deflated and re-inflated with air, during use of the endotracheal tube. As such, the pressure in each of the inner and outer balloons may be regulated to avoid harm to the patient. For instance, inflating the outer balloon with agitated water while the inner balloon is inflated may lead to overpressure on the patient's trachea. Conversely, deflating the inner balloon without compensating by inflating the outer balloon with agitated water may cause the endotracheal tube to become unstable, which can create a risk of migration of the endotracheal tube or of an oxygen shortage to the patient.

In some examples, an injection device may be connected to the inner balloon and the outer balloon to regulate the pressures within both the inner balloon and the outer balloon. The injection device may enable conservation of air volume initially injected into the inner balloon.

In some examples, an injection device may include a body. The body may have a face with a convex surface. A flexible membrane may cover the convex surface of the face thereby forming an expandable balloon. When the interior space of the body is filled with air, the flexible membrane may expand. The interior of the body may be in fluid communication with an inner balloon port. The inner balloon port may be connected to the endotracheal tube such that the inner balloon port is in fluid communication with the inner balloon of the endotracheal tube. The body of the injection device may include a channel in fluid communication with a source port and an outer balloon port. In some examples, the source port may be connected to a source of water, such as a syringe. The outer balloon port may be connected to the endotracheal tube such that the outer balloon port is in fluid communication with the outer balloon of the endotracheal tube.

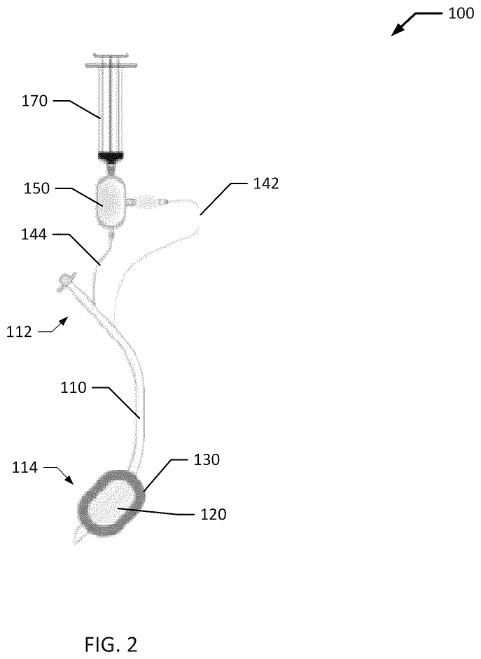

FIG. 2 illustrates an endotracheal tube system 100 according to an example embodiment of the present disclosure. The endotracheal tube system 100 may include an endotracheal tube 110, a first expandable bag 120 disposed on the endotracheal tube 110, and a second expandable bag 130. In some examples, the second expandable bag 130 may surround the first expandable bag 120. In other examples, the second expandable bag 130 may be connected to the first expandable bag 120 in any other suitable form (e.g., partially covering the first expandable bag 120). The endotracheal tube 110 may include a proximal portion 112 and a distal portion 114. In some examples, the first expandable bag 120 and/or the second expandable bag 130 may be disposed on the distal portion 114 of the endotracheal tube 110. In other examples, the first expandable bag 120 and/or the second expandable bag 130 may be disposed on any other suitable portion of the endotracheal tube 110 (e.g., proximal portion 112 or a central portion).

In some examples, the first expandable bag 120 may be configured to contain a first material that can be used to inflate/deflate the first expandable bag 120. In some examples, the first material may include air. In other examples, the first material may be any other suitable material (e.g., any other suitable gas, or any other suitable material).

In some examples, the second expandable bag 130 may be configured to contain a contrast agent. The contrast agent may include any material that may be visible when viewed using an extracorporeal scanner (e.g., ultrasound probe). For example, the contrast agent may be ultrasound-visible. In some examples, the contrast agent may include water or saline (e.g., agitated water or saline).

In some examples, the first expandable bag 120 and/or the second expandable bag 130 are transparent. In other examples, the first expandable bag 120 and/or the second expandable bag 130 are not transparent (e.g., translucent). In some examples, the first expandable bag 120 and/or the second expandable bag 130 may be in a form of a balloon. In other examples, the first expandable bag 120 and/or the second expandable bag 130 may be in any other suitable form as long as it is expandable and as long as it can contain another material, such as the first material or the contrast agent.

In some examples, the endotracheal tube system 100 may further include a first channel 142 and a second channel 142. The first channel 142 may be connected to the first expandable bag 120. In some examples, the first material may be delivered from a source of the first material to the first expandable bag 120 via the first channel 142. For example, the first material may be delivered from a source of the first material (e.g., a syringe filled with the first material) to the first expandable bag 120 via the first channel 142. The second channel 144 may be connected to the second expandable bag 130.

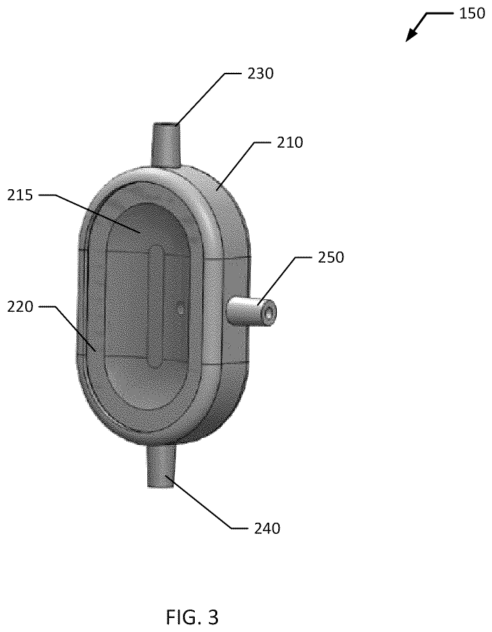

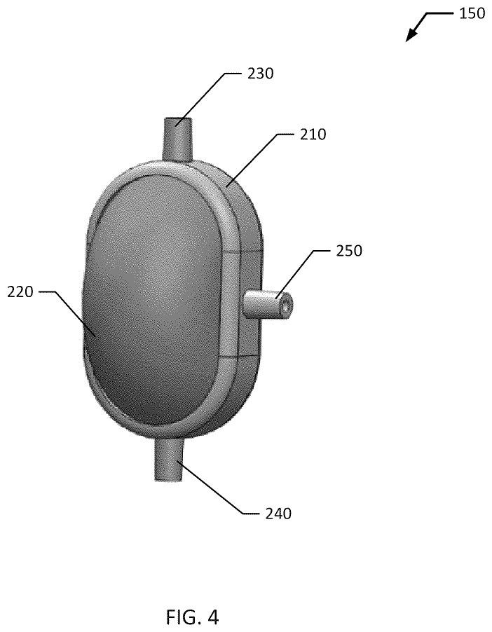

In some examples, the endotracheal tube system 100 may further include an injection device 150. As shown in FIGS. 3 and 4, the injection device 150 may include a body 210 and one or more ports 230, 240, 250 disposed on or in the body 210. In some examples, the body 210 may include an inner (hollow) space 215 and a flexible surface/membrane 220. The flexible surface/membrane 220 may face the inner space 215. The injection device 150 may be in a deflated state (as shown in FIG. 3) when the flexible surface 220 is pressed down towards the inner space 215. The injection device 150 may be in an inflated state (as shown in FIG. 4) when a pressure pressing the flexible surface 220 down towards the inner space 215 is removed.

In some examples, the one or more ports of the injection device 150 may include a first port 230, a second port 240, and a third port 250. The first port 230 may be configured to be in fluid communication with a source 170 of the contrast agent. In some examples, the source 170 of the contrast agent may be a syringe filled with the contrast agent. In other examples, the source 170 of the contrast agent may be any other suitable type of device that can supply the contrast agent to the injection device 150.

The second port 240 may be configured to be in fluid communication with the second expandable bag 130. In some examples, the contrast agent may be delivered from the source 170 to the second expandable bag 130 via the second port 240. In some examples, the second channel 144 may be connected between the second port 240 and the second expandable bag 130, and the contrast agent may be delivered from the second expandable bag 130 via the second channel 144. In some examples, the injection device 150 may be configured to deliver the contrast agent to the second expandable bag 130 while the injection device 150 is transitioning from the deflated state to the inflated state.

In some examples, the third port 250 may be configured to be in fluid communication with the first expandable bag 120. In some examples, the first channel 142 may be connected between the third port 250 and the first expandable bag 120. In some examples, the second port 240 and the third port 250 may be connected to the second expandable bag 130 and the first expandable bag 120, respectively, while ultrasonography is performed on a target patient. This feature will be discussed in more detail below.

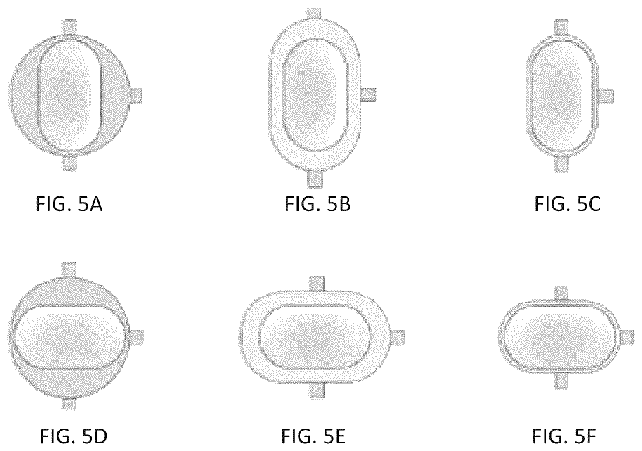

FIGS. 5A-F illustrate various configurations of the injection device 150 according to an example embodiment of the present disclosure. As shown in FIGS. 5A-F, the injection device 150 can be configured in various shapes. In some examples, the body 210 and/or the flexible surface 220 may have a circular or oval shape. In other examples, the body 210 and/or the flexible surface 220 may have any other suitable shape.

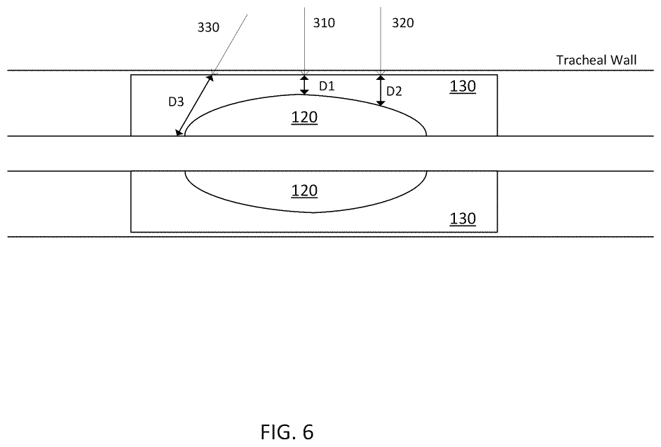

FIG. 6 is a cross-sectional view of the injection device 150 according to an example embodiment of the present disclosure. As shown in FIG. 6, the first expandable bag 120 may be surrounded by the second expandable bag 130. The endotracheal tube 110 may be also surrounded by the first expandable bag 120 and the second expandable bag 130. In FIG. 6, when the first expandable bag 120 and the second expandable bag 130 are inflated, and the second expandable bag 130 may be almost touching the tracheal wall of the patient. In this example, the distance between the outer surface of the first expandable bag 120 and the outer surface of the second expandable bag 130 is different when viewed from different directions, such as a first direction 310 and a second direction 320. That is, the distance D1 (when viewed from the first direction 310) may be smaller than the distance D2 (when viewed from the second direction 320). Therefore, the observation/detection of the first/second expandable bag (e.g., using an ultrasound probe) may also change. Also, in some cases, when the viewing angle is titled, as in the third direction 330, the ultrasound (or any detecting medium) may miss the first/second expandable bag.

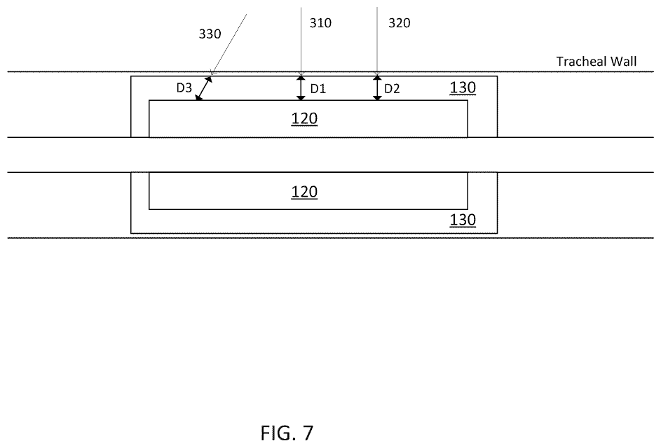

FIG. 7 is a cross-sectional view of the injection device 150 according to another example embodiment of the present disclosure. The injection device of FIG. 7 may address the issue of the injection device of FIG. 6. As shown in FIG. 7, in this example, the outer surface of the first expandable bag 120 may be substantially parallel with the outer surface of the second expandable bag 130, and thus, the distance D1 (when viewed from the first direction 310) is substantially the same as the distance D2 (when viewed from the second direction 320). Also, in this example, the first expandable bag 120 may be more extended to the sides so that when the viewing angle is titled as in the third direction 330, the ultrasound (or any detecting medium) may still not miss the first/second expandable bag, thereby having a broader detection coverage.

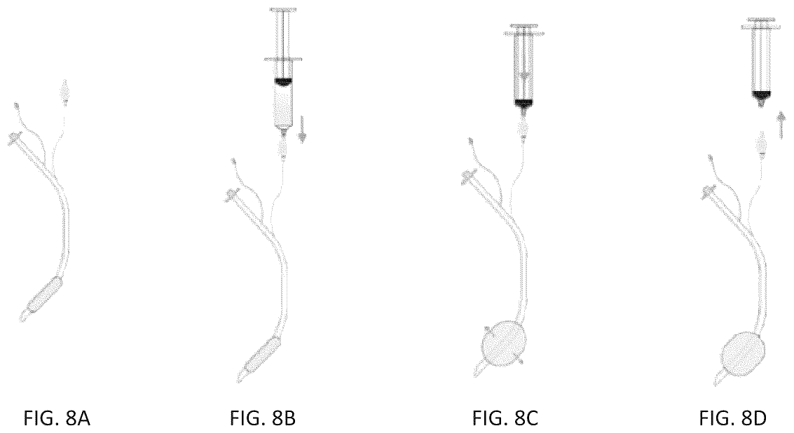

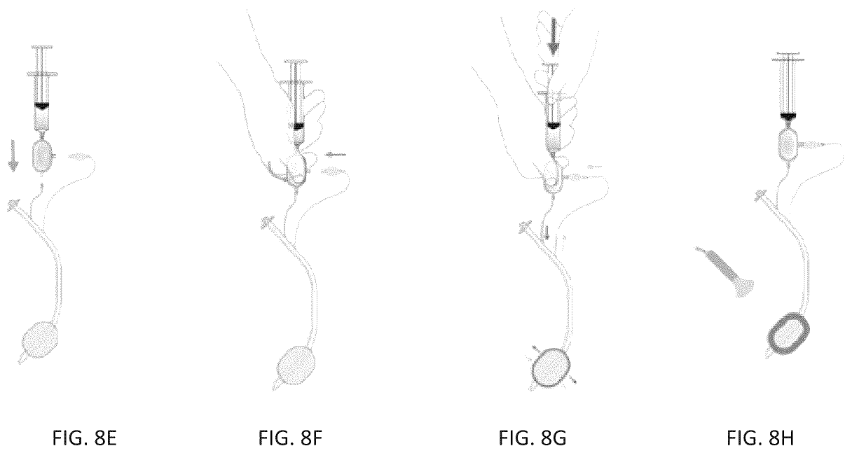

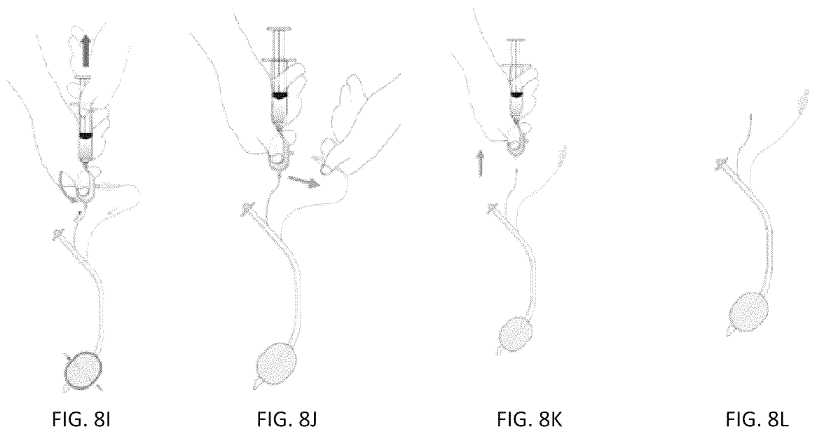

FIGS. 8A-L illustrate a method of inserting the endotracheal tube system into a patient's trachea and using the endotracheal tube system to confirm the location of the endotracheal tube system.

In FIG. 8A, the endotracheal tube system may be provided. Here, the first/second expandable bag may be deflated. In some examples, the endotracheal tube system may be inserted into the patient's trachea in this step (FIG. 8A).

In FIGS. 8B and 8C, a source (e.g., syringe) of the first material (e.g., air) may be connected to the first channel 142, and the first material may be injected into the first expandable bag 120 via the first channel 142, which inflates the first expandable bag 120. In FIG. 8D, the source of the first material may be disconnected from the first channel 142.

In FIG. 8E, the injection device 150 may be connected to the second channel 144 (via the second port 240). At this time, the source 170 (e.g., syringe) of the contrast agent may be also connected to the injection device 150 (via the first port 230).

In FIG. 8F, the injection device 150 (e.g., the flexible surface 220) may be pressed down. In some examples, the injection device 150 (e.g., the flexible surface 220) may be pressed down before the contrast agent is injected into the injection device 150/second channel 144. When the injection device 150 (e.g., the flexible surface 220) is pressed down, the injection device 150 may be in a deflated state. The first channel 142 may not be connected to the injection device 150 before the injection device 150 is in a deflated state. If the second expandable bag 130 is inflated while the first expandable bag 120 is (fully) inflated, this may cause an overpressure on the patient's trachea. In order to prevent the overpressure, the injection device 150 (e.g., the flexible surface 220) may be pressed down before the contrast agent is injected into the injection device 150/second channel 144 and/or before the first channel 142 is connected to the injection device 150. When the injection device 150 is in the deflated state, the first channel 142 may be connected to the injection device (via the third port 250).

In FIG. 8G, the pressure over the injection device 150 (e.g., the flexible surface 220) may be released. The contrast agent may be injected from the source 170 into the injection device 150/second channel 144, thereby inflating the second expandable bag 130. In some examples, the pressure over the injection device 150 (e.g., the flexible surface 220) may be released while the contrast agent is injected from the source 170 into the injection device 150/second channel 144. That is, the injection device 150 may deliver the contrast agent to the second expandable bag 130 while the injection device 130 is transitioning from a deflated state to an inflated state. In some examples, the release of the pressure on the injection device 150 while the first channel 142 is connected to the injection device may remove some first material from the first expandable bag 120, thereby partially deflating the first expandable bag 120. In this way, the overpressure on the patient's trachea may be avoided.

In FIG. 8H, the position of the endotracheal tube 110 may be checked using an extracorporeal scanner. The contrast agent may be visible when viewed using the extracorporeal scanner. In some examples, the extracorporeal scanner may be an ultrasound probe.

After the position of the endotracheal tube 110 is checked, as shown in FIG. 8I, the contrast agent may be removed from the second expandable bag 130, thereby deflating the second expandable bag 130. The source 170 of the contrast agent may receive the contrast agent. At this time, the first expandable bag 120 may be inflated to stabilize the first expandable bag 120. For example, the first material (e.g., air) may be re-injected into the first expandable bag, for example, by pressing down the injection device 150. The inflation of the first expandable bag 120 while removing the contrast agent from the second expandable bag 130 (thereby deflating the second expandable bag 130) may reduce the risk of migration of the endotracheal tube. This may also decrease the pressure on the tracheal wall and tracheal mucosa.

In FIG. 8J, the first channel 142 may be disconnected from the injection device 150. In FIG. 8K, the second channel 144 may be disconnected from the injection device 150. FIG. 8L shows the endotracheal tube 110 with the first expandable bag being inflated after the first and second channels are disconnected from the injection device 150.

Aspects of the present disclosure may have various clinical advantages, such as: rapid, safe, easy confirmation of tracheal intubation or endo-bronchial intubation; possibly done by bedside ultrasound machine that already available in every clinical area; useful in intubation done by non-skillful staff; useful in cases ETCO₂ or CO₂ detector not available; real-time test available during the intubation process. Aspects of the present disclosure also provide various benefits. For example, aspects of the present disclosure may i) prevent misplacement of the endotracheal tube during intubation, thereby avoiding patient desaturation, brain anoxia, and consequent morbidity and mortality; ii) make the system cheap because of easy design and simple cheap materials; iii) decrease X-ray exposure for patients and other health care personnel during the confirmation of endotracheal tube position in the ICU setting; and iv) require less training because of its easiness.

As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

Reference throughout the specification to “various aspects,” “some aspects,” “some examples,” “other examples,” “some cases,” or “one aspect” means that a particular feature, structure, or characteristic described in connection with the aspect is included in at least one example. Thus, appearances of the phrases “in various aspects,” “in some aspects,” “certain embodiments,” “some examples,” “other examples,” “certain other embodiments,” “some cases,” or “in one aspect” in places throughout the specification are not necessarily all referring to the same aspect. Furthermore, the particular features, structures, or characteristics illustrated or described in connection with one example may be combined, in whole or in part, with features, structures, or characteristics of one or more other aspects without limitation.

It is to be understood that at least some of the figures and descriptions herein have been simplified to illustrate elements that are relevant for a clear understanding of the disclosure, while eliminating, for purposes of clarity, other elements. Those of ordinary skill in the art will recognize, however, that these and other elements may be desirable. However, because such elements are well known in the art, and because they do not facilitate a better understanding of the disclosure, a discussion of such elements is not provided herein.

The terminology used herein is intended to describe particular embodiments only and is not intended to be limiting of the present disclosure. As used herein, the singular forms “a,” “an,” and “the” are intended to include the plural forms as well, unless otherwise indicated. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term ‘at least one of X or Y’ or ‘at least one of X and Y’ should be interpreted as X, or Y, or X and Y.

Without further elaboration, it is believed that one skilled in the art can use the preceding description to utilize the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

1. An endotracheal tube system comprising:
an endotracheal tube;
a first expandable bag disposed on the endotracheal tube; and
a second expandable bag surrounding the first expandable bag, wherein the second expandable bag is configured to contain a contrast agent.

2. The endotracheal tube system of claim 1, wherein the first expandable bag is disposed on a distal portion of the endotracheal tube.

3. The endotracheal tube system of claim 1, wherein the first expandable bag is configured to contain air.

4. The endotracheal tube system of claim 1, wherein the contrast agent is ultrasound-visible.

5. The endotracheal tube system of claim 1, wherein the contrast agent comprises agitated water or saline.

6. The endotracheal tube system of claim 1, further comprising an injection device, wherein the injection device comprises:
a body; and
one or more ports on or in the body,
wherein the body comprises:
an inner space; and
a flexible surface facing the inner space, wherein the injection device is in a deflated state when the flexible surface is pressed down towards the inner space.

7. The endotracheal tube system of claim 6, wherein the injection device is in an inflated state when a pressure pressing the flexible surface down towards the inner space is removed.

8. The endotracheal tube system of claim 6, wherein the one or more ports comprises:
a first port configured to be in fluid communication with a source of the contrast agent;
a second port configured to be in fluid communication with the second expandable bag; and
a third port configured to be in fluid communication with the first expandable bag.

9. The endotracheal tube system of claim 8, wherein the injection device is configured to deliver the contrast agent to the second expandable bag while the injection device is transitioning from the deflated state to an inflated state.

10. The endotracheal tube system of claim 8, wherein the second port and the third port are connected to the second expandable bag and the first expandable bag, respectively, while ultrasonography is performed on a target patient.

11. The endotracheal tube system of claim 1, wherein the first and second expandable bags are transparent.

12. A method of inserting the endotracheal tube system of claim 1 into a patient's trachea, the method comprising:
inserting the endotracheal tube in the patient's trachea;
inflating the first expandable bag by injecting a first material into the first expandable bag;
inflating the second expandable bag by injecting the contrast agent into the second expandable bag; and
partially deflating the first expandable bag.

13. The method of claim 12, further comprising checking a position of the endotracheal tube using an extracorporeal scanner, wherein the contrast agent is visible when viewed using the extracorporeal scanner.

14. The method of claim 13, wherein the extracorporeal scanner comprises an ultrasound probe.

15. The method of claim 13, further comprising, responsive to checking the position, deflating the second expandable bag by removing the contrast agent from the second expandable bag, and inflating the first expandable bag, thereby stabilizing the first expandable bag.

16. The method of claim 12, wherein the first material comprises air.

17. The method of claim 12, wherein the first expandable bag is partially deflated while the second expandable bag is inflated, thereby preventing overpressure on the patient's trachea.

18. The method of claim 12, wherein the first expandable bag is disposed on a distal portion of the endotracheal tube.

19. The method of claim 12, wherein the contrast agent comprises agitated water or saline.

20. The method of claim 12, wherein the endotracheal tube system further comprises an injection device, wherein the injection device comprises:
a body; and
one or more ports on or in the body,
wherein the body comprises:
an inner space; and
a flexible surface facing the inner space, wherein the injection device is in a deflated state when the flexible surface is pressed down towards the inner space.
